(19) United States
(12) Patent Application Publication
(10) Pub. No.: US 2011/0313264 A1
(43) Pub. Date: Dec. 22, 2011

(54) FULL BODY PLETHYSMOGRAPHIC CHAMBER INCORPORATING PHOTOPLETHYSMOGRAPHIC SENSOR FOR USE WITH SMALL NON-ANESTHETIZED ANIMALS

(75) Inventor: Bernard F. Hete, Kittanning, PA (US)

(73) Assignee: STARR LIFE SCIENCES CORP., Oakmont, PA (US)

(21) Appl. No.: 12/968,273

(22) Filed: Dec. 14, 2010

Related U.S. Application Data
(60) Provisional application No. 61/286,368, filed on Dec. 14, 2009.

(51) Int. Cl. A61B 5/1455

(52) U.S. Cl. 600/340

(57) ABSTRACT

A full body plethysmographic chamber includes a noninvasive photoplethysmographic sensor within the chamber. The noninvasive photoplethysmographic sensor is for mobile animals such as small rodents, namely rats and mice in the full body plethysmographic chamber and is useful in a laboratory research environment. The noninvasive photoplethysmographic sensor may be a neck clip or collar which provides an easily affixed attachment mechanism. The neck location will provide significant blood flow under all conditions and also offers inherent bite resistance to the sensor platform. The system of the present invention provides a commutator for the sensor wires for allowing untwisting of the system wires.

The present invention claims priority of U.S. Provisional Patent Application Ser. No. 61/286,368 entitled “Photoplethysmographic Sensor for use in Full Body Plethysmographic Chamber on Small Non-Anesthetized Animals” filed Dec. 14, 2009.

### BACKGROUND OF THE INVENTION

1. Field of the Invention

The present invention relates to photoplethysmographic readings for animal research and more particularly, the present invention is directed to a full body plethysmographic chamber incorporating a noninvasive photoplethysmographic sensor for mobile animals such as small rodents.

2. Background Information

In general, medical research begins on animals, and among the animals used in research, teaching, and testing, mice and rats comprise, by far, the largest majority of all experimental mammals. Accurate global figures for animal testing are difficult to obtain, or to verify. The Nuffield Council on Bioethics reports that global annual estimates range from 50 to 100 million animals. Others have estimated that 30-60 million mice are used for research purposes annually. In 2006, the University of California Center For Animal Alternative conservatively estimated that that well over 6,000,000 mice were used annually for research purposes in the United States alone. Regardless of the actual annual number of small mammals used in research, the number is considerable and there is a growing need for improved tools to assist in this research.

The remarkable genetic similarity of mice to humans, combined with great convenience of the animals, generally accounts for mice so often being the experimental model of choice in research. Mice are widely considered to be the prime model of inherited human disease. In addition to being genetically similar to humans, mice are small and inexpensive to maintain, are widely availability, have a relatively low cost, provide ease of handling, and fast reproduction rate. Rats represent the next largest majority of experimental mammals, with the number of rats used annually for research being estimated as ⅓-¼ the number of mice. As further evidence of the importance of mice in research, the 2007 Nobel Medicine Prize was awarded to Mr. Capecii, Mr. Evans, and Mr. Smithies for the development of “Knockout Mice” which have been designated as the test-bed of biomedical research for the 21^{st} century.

Small Animal Noninvasive Pulmonary Research

Measurements of breathing rates, breathing patterns, lung volumes, trans-pulmonary pressure, lung mechanics and gas exchange in small mammals, have been critically important to research. The ability to determine, in vivo, the respiratory function in laboratory or research mice is of great interest because of the prominent role played by these animals in biomedical, pharmacological and toxicological research. For example, mice are, at present, the preferred species used as an experimental model of allergic airway disease. This is largely due to a number of advantages including a well characterized genome and immune system, short breeding periods, the availability of inbred and transgenic strains, suitable genetic markers, the ability to readily induce genetic modifications and pragmatically, relatively low maintenance costs as discussed above. The development of viable mouse models has largely contributed to a better understanding of the patho-mechanisms underlying allergic airway inflammation and airway hyper-responsiveness.

A variety of non-invasive measures of pulmonary function for small mammals have been developed such as impedance pneumography which uses various circumferential strain-gauge respirometers for the subject and body plethysmography which represents the most commonly used technique currently.

A plethysmograph is an instrument for measuring changes in volume within an organ, which, usually resulting from fluctuations in the amount of blood or air that the organ contains. A body plethysmography can include very sensitive lung measurement used to detect lung pathology that might be missed with conventional pulmonary function tests. In human subjects this method of obtaining the absolute volume of air within the subject's lungs may also be used in situations where several repeated trials are required or where the subject is unable to perform the multi-breath tests. The technique in humans requires moderately complex coaching and instruction for the subject. In the USA, such tests are usually performed by Certified or Registered Pulmonary Function Technologists (CPFT or RPFT) who are credentialed by the National Board for Respiratory Care. More specifically, the test is done by enclosing the subject in an airtight chamber often referred to as a body box; a pneumotachometer is used to measure airflow while a mouth pressure transducer with a shutter measures the alveolar pressure. The most common measurements made using body plethysmographs are thoracic gas volume (VTG) and airway resistance (R_{AW}). This test is used mainly in the Pulmonary Function Testing laboratories.

For animal studies on small mammals the pulmonary research solutions offered by Buxco (www.Buxco.com) are representative of a class of plethysmographic devices for small animals including the full body plethysmographic chambers for small mammals. The Buxco devices include unrestrained whole or full body plethysmographic chambers, specialized sealed restraint tubes, and even a head out partial body plethysmographic chamber device for monitoring respiratory and related functions in small animals. These research devices are generally associated with full body plethysmography generally for assessing lung function. This approach to assess lung function involves placing the subject into a small completely sealed chamber and measuring the pressure changes within the chamber that occur as the animal breathes. The animal can thus be conscious and unrestrained. This technique currently enjoys wide popularity. It is critical that the system remain sealed to obtain reliable measurements in the chamber. Venting in precisely controlled manners is also known with such sealed plethysmographic chambers. Within the meaning of this application a full body plethysmographic chamber is a sealed chamber that receives the entire body of the subject and is configured to measure physiologic parameters from the chamber such as pressure variations.

The limitation in the existing systems is that they have not allowed for additional parameters to be obtained from the animal in the chamber. See the 2003 study from University of Washington Medical Center regarding “Oxygen regulation and limitation to cellular respiration in mouse skeletal muscle in vivo” in which the researchers describe the development of “novel” methodology for measuring oxygen consumption in the subject mice. See also the 1998 study from Case Western Reserve regarding the “Altered respiratory responses to hypoxia in mutant mice deficient in neuronal nitric oxide synthase” wherein the experiments were conducted on awake and anaesthetized mutant and wild-type control mice. For the study of the un-anesthetized subjects the physiologic parameters were limited to the whole body plethysmograph described above and monitoring of oxygen consumption and carbon dioxide production from gas monitoring of the chamber vent.

As further background, an article entitled “A compact respiratory-triggering device for routine micro-imaging of laboratory mice” in the Journal of Magnetic Resonance Imaging Volume 8 issue 6 pages 1343-1348 Dec. 12, 2005 by Kevin R. Minard, PhD, Robert A. Wind, PhD, Robyn L. Phelps, PhD discloses a partial-body plethysmograph that was developed for measuring the respiratory flow of anesthetized mice during routine micro-imaging experiments performed in the close confines of an 89-mm-diameter, vertical-bore magnet. For a more comprehensive overview of the study of pulmonary function in mice see the article entitled “Invasive and noninvasive methods for studying pulmonary function in mice” by Thomas Glaab, Christian Taube, Armin Braun and Wayne Mitzner in the Respiratory Research 2007, 8:63doi:10.1186/1465-9921-8-63 which describes various “classical and recent” methods of measuring airway responsiveness in vivo including both invasive methodologies in anesthetized, intubated mice (repetitive/non-repetitive assessment of pulmonary resistance (R_{L}) and dynamic compliance (C_{dyn}); measurement of low-frequency forced oscillations (LFOT)) and noninvasive technologies in conscious animals (head-out body plethysmography; barometric whole-body plethysmography).

For further background see 1. Irvin C G, Bates J H: Measuring the lung function in the mouse: the challenge of size. Respir Res 2003, 4:4; 2. Lorenz J N: A practical guide to evaluating cardiovascular, renal, and pulmonary function in mice. Am J Physiol Regulatory Integrative Comp Physiol 2002, 282:R1565-1582; 3. Bates J H, Irvin C G: Measuring lung function in mice: the phenotyping uncertainty principle. J Appl Physiol 2003, 94:1297-1306; 4. Hoymann H G: Invasive and noninvasive lung function measurements in rodents. J Pharmacol Toxicol Methods 2007, 55:16-26; 5. Lofgren J L S, Mazan M R, Ingenito E P, Lascola K, Seavey M, Walsh A, Hoffman A M: Restrained whole body plethysmography for measure of strain-specific and allergen-induced airway responsiveness in conscious mice. J Appl Physiol 2006, 101:1495-1505; 6. Irvin C G, Peslin R: Methods for measuring total respiratory impedance by forced oscillations. Bull Eur Physiopathol Respir 1986, 22:621-631; 7. Hamelmann E, Schwarze J, Takeda K, Oshiba A, Larsen G L, Irvin C G, Gelfand E W: Noninvasive measurement of airway responsiveness in allergic mice using barometric plethysmography. Am J Respir Crit Care Med 1997, 156:766-775; 8. Chong B T, Agarwal D K, Romero F A, Townley R G: Measurement of bronchoconstriction using whole-body plethysmograph: comparison of freely moving versus restrained guinea pigs. J Pharmacol Toxicol Methods 1998, 39:163-168.

There is a need to assist researchers for conducting respiratory research on animals, such as on rats and mice, to allow for direct measurements of physiologic cardio-pulmonary parameters. It is an object of the present invention to provide a wider variety of easily utilized tools to the animal researcher.

Photoplethysmographic Sensor

A photoplethysmograph is an optically obtained plethysmograph, which, generically, is a measurement of changes in volume within an organ whole body, usually resulting from fluctuations in the amount of blood or air that the organ contains. A common photoplethysmograph is obtained by using a pulse oximeter. A conventional pulse oximeter monitors the perfusion of blood to the dermis and subcutaneous tissue of the skin. Pulse oximetry is a non invasive method that allows for the monitoring of the oxygenation of a subject's arterial blood, generally a human or animal patient or an animal (or possibly human) research subject. The patient/research distinction is particularly important in animals where the data gathering is the primary focus, as opposed to care-giving, and where the physiologic data being obtained may, necessarily, be at extreme boundaries for the animal.

As a brief history of pulse oximetry, reportedly Matthes developed in 1935 the first 2-wavelength earlobe O₂ saturation meter with red and green (later switched to red and infrared) filters. Further, in 1949, an inventor Wood added a pressure capsule to squeeze blood out of the earlobe to obtain zero setting in an effort to obtain absolute O₂ saturation value when blood was readmitted. The concept is similar to today's conventional pulse oximetry but suffered due to unstable photocells and light sources and the method was not used clinically. In 1964 an inventor Shaw assembled the first absolute reading ear oximeter by using eight wavelengths of light, and this design was commercialized by Hewlett Packard. This use was limited to pulmonary functions.

Effectively, modern pulse oximetry was developed in 1972, by Aoyagi at Nihon Kohden using the ratio of red to infrared light absorption of pulsating components at the measuring site, and this design was commercialized by BIOX/Ohmeda in 1981 and Nellcor, Inc. in 1983. Prior to the introduction of these commercial pulse oximeters, a patient's oxygenation was determined by a painful arterial blood gas, a single point measure which typically took a minimum of 20-30 minutes processing by a laboratory. It is worthy to note that in the absence of oxygenation, damage to the human brain starts in 5 minutes with brain death in a human beginning in another 10-15 minutes. Prior to its introduction, studies in anesthesia journals estimated US patient mortality as a consequence of undetected hypoxemia at 2,000 to 10,000 deaths per year, with no known estimate of patient morbidity. Pulse oximetry has become a standard of care for human patients since about 1987.

Pulse oximetry has been a critical research tool for obtaining associated physiologic parameters in humans and animals beginning soon after rapid pulse oximetry became practical.

In conventional pulse oximetry a sensor is placed on a thin part of the subject's anatomy, such as a human fingertip or earlobe, or in the case of a neonate, across a foot, and two wavelengths of light, generally red and infrared wavelengths of light, are passed from one side to the other. Changing absorbance of each of the two wavelengths is measured, allowing determination of the absorbances due to the pulsing artery alone, excluding venous blood, skin, bone, muscle, fat, etc. Based upon the ratio of changing absorbance of the red and infrared light caused by the difference in color between oxygen-bound (bright red) and oxygen unbound (dark red or blue, in severe cases) blood hemoglobin, a measure of oxygenation (the percent of hemoglobin molecules bound with oxygen molecules) can be made. The measured signals are also utilized to determine other physical parameters of the subjects, such as heart rate (pulse rate).

Starr Life Sciences, Inc. has utilized pulse oximetry measurements to calculate other physiologic parameters such as breath rate, pulse distension, and breath distention, which can be particularly useful in various research applications.

Regarding human and animal pulse oximetry, the underlying theory of operation remains the same. However, consideration must be made for the particular subject or range of subjects in the design of the pulse oximeter, for example the sensor must fit the desired subject (e.g., a medical pulse oximeter for an adult human finger simply will not adequately fit onto a mouse finger or paw; and regarding signal processing the signal areas that are merely noise in a human application can represent signals of interest in animal applications due to the subject physiology). Consequently there can be significant design considerations in developing a pulse oximeter for small mammals or for neonates or for adult humans, but, again the underlying theory of operation remains substantially the same.

In addressing animal pulse oximetry, particularly for small rodents, one approach has been to modify existing human or neonate oximeters for use with rodents. This approach has proven impractical as the human based systems can only stretch so far and this approach has limited the use of such adapted oximeters. For example, these adapted human oximeters for animals have an upper limit of heart range of around 400 or 450 beats per minute which is insufficient to address mice that have a conventional heart rate of 400-800 beats per minute. Starr Life Sciences has developed a small mammal oximeter, rather than an adapted human model, that has effective heart rate measurements up to and beyond 1000 beats per minute, and this is commercially available under the Mouse Ox™ oximeter brand.

Neck Collars

In animal fields, neck collars have served as a mounting platform for selected sensors, such as bark sensors or position sensors in animal control collars that direct a pressure pulse wave to an animal as a negative stimulus to deter undesired behavior (e.g. shock), such as described in U.S. Pat. No. 6,830,013. Other animal control collars use a collar mounted sensor sensing a perimeter wire for animal control, see U.S. Pat. No. 6,657,544 and also products sold under the brand Invisible Fence®.

In wildlife research, collars are the most common form of transmitter attachment for mammals in radio-telemetry studies, often wildlife studies. The following discussion offers background information on such radio-telemetry collar mounting considerations. Collars should be made of materials which are durable; are comfortable and safe for the animal; can withstand extreme environmental conditions; do not absorb moisture; and maintain their flexibility in low temperatures. Common collar materials for transmitter mounting in radio-telemetry based studies are butyl belting, urethane belting, flat nylon webbing, tubular materials, metal ball-chains, PVC plastic, brass or copper wire and cable ties. The transmitter package may be situated either under the animal's neck or on top of it. Collars must be properly fitted for the comfort and safety of the animal.

A collar should fit snugly to prevent it coming off or chafing the animal as it moves, but it must also be sufficiently loose as to be comfortable and not interfere with swallowing or panting. To reduce the risk of chafing on the neck, collars should generally be fastened at the side, with any metal fittings covered or smoothed on the inside surface of the collar. Neck circumference will vary according to species, age, sex and sometimes the season. Transmitter manufacturers usually have records of collar sizes previously used for various species. Collar thickness and width are important considerations. Width of the collar will affect how the transmitter sits on the animal's neck. Some researchers prefer narrower collars because there is less surface area in contact with the animal. Others prefer wider collars for better weight distribution. One of the most important considerations in collar designs for roaming wildlife should be the possibility of the collar getting caught up in vegetation. This is a particularly important consideration with small mammals (especially those that burrow). Expandable collars and harnesses are mandatory in those cases where it is necessary to allow for growth in young animals or for species which undergo neck swelling. Braided nylon over surgical tubing and nylon web with elastic folds are offered as expandable collars by one company. Expandable collars should not be used unless they are well tested, as poorly designed collars can be very problematic. In the past, certain collars have stretched prematurely as a result of social interactions or behaviors such as neck rubbing. As a result, there is always the possibility of transmitter loss, icing up in winter, or of the collar becoming snagged by branches or even the animal's own legs.

Breakaway or “rot-away” collars are strongly recommended in cases where the researcher does not intend to recapture the animal and remove the collar. Breakaway collars or harnesses incorporate a link of material which is designed to break away and allow the transmitter to drop off after a pre-determined interval. Breakaway links should be environmentally degradable material or electronic links controlled by timers or radio receivers. Environmentally degradable materials which have been used for this purpose include cotton thread and sections of cotton fire hose or cotton spacers on large mammal collars. These weak links may also function to break and free the animal if the collar/harness is snagged on a branch. However, it is important to consider that the breakaway collar or harness does not impair the movement or activities of the animal during the period in which it is being shed. For example, a breakaway bird body harness could easily impair wing movement as it is lost and result in mortality. Radio and timer-controlled breakaways may be jammed by freezing or dirt, and also add to the size, weight and complexity of the transmitter package. Where appropriate, it is recommended that collars and harnesses be marked in order to enhance their visibility. Paint or non-metallic reflective materials may be sewn or glued to collars and harnesses; however, this is likely not appropriate for cryptic species. Metallic tape or foils should not be used as they will detune the transmitting antenna. Adhesive tapes should also not be used as they are not very durable and may foul fur or feathers. For game species or urban studies it may also be helpful to mark a contact phone number on the collar. Color-coded collars are also available from telemetry equipment manufacturers. VHF temperature sensors may be used to monitor either the animal's body temperature or the environmental temperature. Body temperature data may be useful in determining health or reproductive status, and ambient temperature may also be utilized for habitat selection or hibernation studies. Transmitters for body temperature may be placed subcutaneously, internally such as within the inner ear. Transmitters for ambient or den temperature may be placed on a regular collar or harness. Size or weight limitations and the data precision required will also affect transmitter type and placement.

A 2003 study at Kansas State University entitled “Wearable Sensor System for Wireless State-of-Health Determination in Cattle” disclosed a collection of sensors for animal research which was designed to incorporate off-the-shelf and custom-designed sensors and modules to provide cost-effective animal health monitoring capabilities. These sensors and modules included a GPS (Global Positioning System) unit, a pulse oximeter, a core body temperature sensor, an electrode belt, a respiration transducer, and an ambient temperature transducer. A GPS collar unit was intended to yield both animal location and movement data. A commercial CorTemp system was intended to monitor core body temperature continuously via an ingestible bolus. The bolus wirelessly transmitted temperature data to a receiving unit connected to BMOO. The animal was also to wear a Polar electrode belt that acquires pulse rate and transmits it wirelessly to the core body temperature receiving unit. A custom-designed pulse oximeter was proposed to measure blood oxygen saturation and pulse rate from an ear tag that the animal would wear. It is interesting to note that for pulse oximetry in cattle, off the shelf human oximeters were insufficient and a custom design was required.

None of the above solutions adequately address laboratory animal research applications using mobile animals in a full body plethysmographic chamber and more particularly, the prior art fails to adequately provide an efficient and noninvasive photoplethysmographic sensors for mobile animals such as small rodents, namely rats and mice in a full body plethysmographic chamber.

It is an object of the present invention to address the deficiencies of the prior art discussed above and to do so in an efficient, cost effective manner.

### SUMMARY OF THE INVENTION

The various embodiments and examples of the present invention as presented herein are understood to be illustrative of the present invention and not restrictive thereof and are non-limiting with respect to the scope of the invention.

One aspect of the invention provides a full body plethysmographic chamber including a noninvasive photoplethysmographic sensor within the chamber. The full body plethysmographic chamber according to the invention further includes an electrical line commutator with wires extending from the electrical line commutator to the photoplethysmographic sensor configured for allowing rotation of the wires. The full body plethysmographic chamber according to one aspect of the invention provides that the photoplethysmographic sensor is configured to be mounted on the neck of the animal. The full body plethysmographic chamber according to one embodiment of the invention provides that a sealed bearing is positioned between the commutator and the photoplethysmographic sensor, and wherein the wires extending from the commutator to the photoplethysmographic sensor extends through the sealed bearing. The full body plethysmographic chamber according to one embodiment of the invention further includes at least one fluid/gas line extending to the animal within the chamber, wherein the fluid/gas line extends through the sealed bearing and with a fluid line commutator coupled to the fluid lines. The full body plethysmographic chamber according to one embodiment may further include at least one additional physiologic sensor coupled to the animal. The full body plethysmographic chamber according to one embodiment is provides such that each physiologic sensor and each fluid line can be selectively coupled and decoupled from the sealed bearing.

One embodiment of the invention provides a method of obtaining physiologic parameters of an animal comprising the steps of: Placing the animal in a sealed full body plethysmographic chamber; Obtaining pulmonary parameters of the animal from the body plethysmographic chamber with a first sensor platform; and Simultaneously obtaining pulse oximetry data from a photoplethysmographic pulse oximetry sensor coupled to the animal.

One aspect of the invention provides a full body plethysmographic chamber including a sealed chamber for receiving an animal; a sensor platform coupled to the chamber for obtaining pulmonary parameters of the animal; at least one additional physiologic sensor coupled directly to the animal within the chamber; and an electrical line commutator with wires extending from the electrical line commutator to each of the physiologic sensors that are coupled directly to the animal with the commutator configured for allowing rotation of the wires. The full body plethysmographic chamber according to the invention may provide that one physiologic sensor that that is coupled directly to the animal is a photoplethysmographic sensor.

According to one non-limiting embodiment of the present invention, a noninvasive photoplethysmographic sensor for mobile animals such as small rodents, namely rats and mice, in a full body plethysmographic chamber is provided.

A noninvasive photoplethysmographic sensor for mobile animals such as small rodents, namely rats and mice in a full body plethysmographic chamber is useful such as in a laboratory research environment. The noninvasive photoplethysmographic sensor may be a neck clip or collar which provides an easily affixed attachment mechanism. The neck location will provide significant blood flow under all conditions and also offers inherent bite resistance to the sensor platform. The system of the present invention provides a commutator for the sensor wires for allowing untwisting of the system wires.

These and other advantages of the present invention will be clarified in the description of the preferred embodiments taken together with the attached figures.

### BRIEF DESCRIPTION OF THE DRAWINGS

FIG. 1 is a schematic representation of a full body plethysmographic chamber incorporating a noninvasive photoplethysmographic sensor for mobile animals such as small rodents, namely rats and mice, in accordance with one embodiment of the present invention; and

FIG. 2 is a schematic representation of a full body plethysmographic chamber incorporating a noninvasive photoplethysmographic sensor for mobile animals such as small rodents, namely rats and mice, in accordance with a further embodiment of the present invention.

### DESCRIPTION OF THE PREFERRED EMBODIMENTS

In summary, the present invention relates to a noninvasive plethysmographic chamber system 10 incorporating a photoplethysmographic sensor 30 for mobile animals, such as rats and mice 14 that are utilized in a sealed full body plethysmographic chamber 12. Photoplethysmographic measurements on laboratory animals have most often been accomplished on restrained and/or anesthetized animals. This limits the research that can be conducted. Further, in the pulse oximetry field there has been a lack of adequate photoplethysmographic sensors for small mice (and even small rats), until the advent of the Mouse Ox™ brand pulse oximeters by Starr Life Sciences. Prior to this development, commercially available pulse oximeters could provide heart rate data up to about 350 or 450 beats per minute (and even this range required special software modifications for some sensors), which were basically suitable for rats but not small mice given that the small mouse will have heart rates in the range of 400 to 800 beats per minute. The Mouse Ox™ brand of pulse oximeters for small rodents has an effective range up to, currently, beyond 1000 beats per minute which has opened up a wider selection of subjects for this type of research.

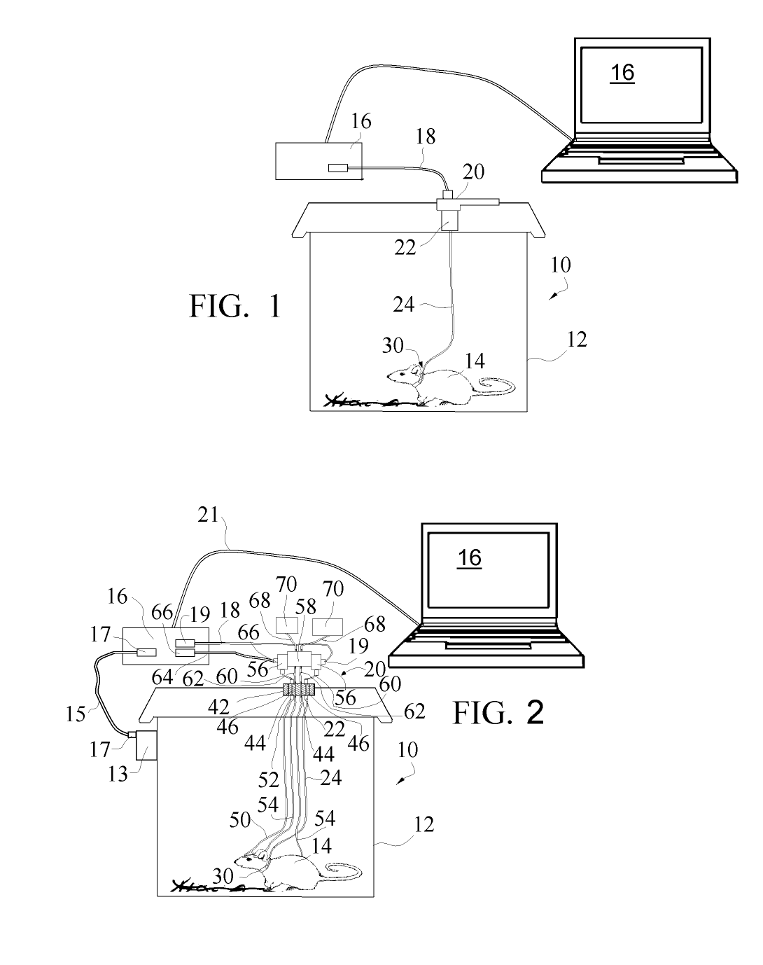

FIG. 1 is a schematic representation of a full body plethysmographic chamber system 10 incorporating a noninvasive photoplethysmographic sensor 30 for mobile animals such as small rodents, namely rats and mice, in a full body plethysmographic chamber 12 in accordance with one embodiment of the present invention.

The general details of the chamber 12 will be well known to those of ordinary skill in animal research fields, and representative examples of which are found from commercially available BUXCO devices. In general the chamber 12 includes a sensor bank or platform 13 obtaining physiologic parameters of the sealed chamber 12, generally pressure variations. However other measurements are possible, particularly with controlled venting and sampling of the interior of the chamber 12. The sensor bank 13 is connected to a controller 16 through cable 15 and couplers 17. The plug in connectors or couplers 17 allow a single controller 16 to be easily disconnected and moved to other chambers 12. The controller 16 may have a separate display as shown, such as a lap top or the like. The general operation and control of a full body plethysmographic chamber 12 is known in the art.

The subject animal may be any animal for which photoplethysmographic measurements are desired. The present invention has particular application to research associated with rats and mice. More accurately the present invention provides particular advantages and expands potential research possibilities when utilized with subjects of the order rodentia, and even more precisely, when utilized with the sub-order muroidia. A particularly advantageous aspect of the present invention is that the system 10 allows for photoplethysmographic measurements from a mobile animal in a full body plethysmographic chamber 12, but the animals will generally still have a certain range of motion therein. However, there is nothing that prevents the system 10 from being effectively utilized for restrained and/or anesthetized animals 14.

As noted in connection with the chamber 12 and sensor bank 13, the system 10 will include a processor or controller 16 coupled thereto. The controller 16 is shown schematically in FIG. 1 and can be formed as a component of a laptop or desktop computer or as an added plug-in accessory thereto (as shown) through cable 21. The controller 16 may be the combination of stand alone hardware and software that is coupled via cable 21 with a computer for the user interface, the display, the memory and for some computation or additional data processing. The present invention also contemplates separate controllers 16 for the sensor bank 13 of the chamber 12 and the sensor 30 (and other sensors 50), however a single integrated controller 16 with separate processing components as shown is acceptable. Preferably, the controller 16 incorporates the commercially available MouseOx™ product from Starr Life Sciences for control and processing of the sensor 30, with the unique sensor mounting and coupling as described hereinafter. The details of the controller 16 for sensor 30 (or other conventional sensors 50 or components or the like), including the user interface, the user display, memory or the like is not discussed herein in detail and are known to those of ordinary skill in the art.

A conventional controller cable 18 extends from the controller 16 for transmitting control and power signals from the controller 16 and data back to the controller 16. As shown in FIG. 2, plug in connectors or couplings 19 allow for easy attachment and release of the controller 16.

In the embodiment of FIG. 1, the controller cable 18 is coupled to a sealed rotation coupling 20, which may also called a swivel link, slip ring or commutator. A mercury swivel commutator, such as available from Mercotac Inc, is a commutator that has been used in electrophysiological experiments involving moving animals. Another acceptable coupling 20 is a commutator available from Plastics One. Any acceptable rotation coupling 20 can be used that transmits the signals with minimal commutator noise and which avoids twisting of the wires, provided that the commutator is or can be sealed so as not to corrupt the chamber 12 operation.

A collar cable 24 is attached to and extends from the rotation coupling 20 through attachment plug in connector or coupling 22 in FIG. 1 to a neck collar or neck clip photoplethysmographic pulse oximetry sensor 30. The rotation coupling 20 allows relative rotation between the controller cable 18 and the collar cable 24. The rotation coupling 20 provides a convenient location for mounting to the chamber 12. The use of the swivel link or rotation coupling 20 allows the animal, e.g. mouse 14, to be effectively freely roaming within the area of the chamber 12, wherein twisting of the cables is avoided. The swivel link or rotation coupling 20, namely the sealed bearing, also serves to effectively divide the system 10 into an animal specific sensor 30 and the controller 16, whereby the controller 16 can be easily used with a large number of animal specific portions in a serial fashion. Further, it allows for easy replacement of the neck clip sensors 30 which is anticipated to have a shorter life span than the controller 16.

The present invention does anticipate that the controller 16 may be simultaneously (e.g. a parallel attachment) connected to a number of animal specific sensors 30 through separate cables 18 to allow for obtaining numerous study results at the same time, but this configuration does not eliminate the advantages of the coupling 20.

The system 10 includes neck clip sensors 30 in the form of emitters and detectors mounted on the neck clip, or on a body encircling collar configured to encircle around the neck of the subject animal. The neck of small mammals such as rats and mice allows for a number of advantages for photoplethysmographic pulse oximetry measurements. The necks of animals of the sub-order muroidia tend to allow for both transmissive and reflective pulse oximetry measurements. Transmissive pulse oximetry is where the received light is light that has been transmitted through the perfuse tissue, whereas in reflective pulse oximetry the representative signal is obtained from light reflected back from the perfuse tissue. Each technique has its unique advantages. Transmissive techniques often result in a larger signal of interest, which is very helpful in small animals that have very small quantities of blood being measured to begin with. Reflective techniques can be used in environments that do not allow for transmissive procedures (e.g. the forehead of a human).

Further, the neck region of the animal offers an area with a relatively large blood flow for the animal, which will improve the accuracy of the measurements. In addition to increased blood flow, the blood flow is present under substantially all conditions. In other areas of the animal, such as the legs, paws and tail, the animal will often cut off blood flow under a variety of conditions. For example if the animal is cold or sufficiently agitated the blood flow to the tail can be shunted. The neck, in contrast represents an area of the animal that will always maintain a constant blood flow for measurements.

The neck location also provides a bite proof location for the sensor mounting. In attempting to remove the sensors the biting of most animals, particularly animals of the sub-order muroidia, will be stronger than the clawing, and the neck location prevents the biting attacks as the animal cannot reach the clip or collar. A secured clip or collar cannot be removed by the animals paws or clawing.

The form and material of the clip or collar for sensor 30 can be any of a wide variety of materials and shapes. The sensors 30 include at least one emitter on the clip or collar configured to be mounted adjacent the subject animal, mouse 14, with each emitter having two light sources of distinct wavelengths; and a receiver on the clip or collar configured to be mounted adjacent the subject mammal for detecting light from the emitter that has been toward tissue of the subject mammal. The emitter and receiver may be configured for transmissive operation or even reflective operation.

The coupling 20 must be a sealed coupling as it enters the chamber 12. FIG. 1 illustrates an embodiment using a sealed commentator at the chamber 12 boundary. An alternative arrangement is shown in FIG. 2 the coupling 20 includes the collection of an electrical line commutator 56, fluid line commutator 58, and sealed bearing 42 and connections 60, 62 there between. The system 10 in the embodiment of FIG. 2 separates the function of the coupling 20 into a sealing function that occurs at the chamber boundary with the sealed bearing 42 and a rotation compensating component provided by the electrical line commutator 56 and the fluid line commutator 58 positioned spaced from (above) the chamber 12 boundary to provide the swivel linkage needed. The separate sealed bearing construction allows for multiple benefits. First is that it allows non-sealed commercially available commutators 56 and 58 to be used. As will be described below it allows for a simple sealing construction for the bearing and allows for various sensors and fluid lines to be utilized in a variety of user designated implementations

The system 10 of the embodiment of FIG. 2 further contemplates additional electrical sensors 50 to be used, such as Eeg, Emg, Ecg, Eog, Ekg leads, temperature sensors, or other desired electrical sensing elements/sensors coupled to the animal 14. Additionally the system 10 accommodates gas/fluid lines 54 to be introduced into the chamber 12. The gas/fluid lines 54 can be used to supply test materials directly to the subject (e.g., intravenous injection site), or to assist in testing physiologic parameters (such as pressure in a blood pressure cuff), or supply respiratory gases that cannot be dispersed in the chamber 12. The possible uses for lines 54 listed here are not exhaustive and are expected to be developed by the researchers as the present system 10 is designed as a tool for expanding the research possibilities.

The sealed bearing 42 will have a stationary outer race and a rotating inner plate with sealed bearings (not shown) there between. The rotating plate is designed with fluid/gas lines 44 extending there through and with electrical lines 46 extending there through in a sealed fashion. Within the chamber 12 the lines 54 couple onto lines 44 which form attaching nubs on either side of the bearing. If one (or both) lines 54 is not needed a cap (not shown) (or caps) can be placed one or the other lines 44 either inside or outside of the chamber 12 to seal the lines 44 for operation of the chamber 12. The sensor 50 will include a plug in connector or coupling 52 to connect to lines 46 inside of chamber. The sensor 30 and cable 24 includes the plug in connector or coupling 22 that attaches to line 46. The use of plug in connectors 22 and 52 allow for easy replacement and removal of sensors 50 and 30.

The dual fluid lines 54 require the use of a fluid commutator 58 such as available from Instech. Lines 62 attach to nubs of line 44 and couple to fluid commutator 58. Lines 68 extend from the fluid commutator 58 to respective sources of fluid/gas 70, if used. This arrangement together with the coupling 20 allows for the untwisting of the wires and fluid lines in a simple manner.

The unwinding of the wires and fluid lines may be done manually or through some automated mechanism. Some automatic mechanisms include tether technologies. Known tether technologies may be on cables 24 line 50 and lines 54 as torsion transmission mechanisms and as bite protecting members. These conventional torsion transmitting members are not shown for clarity.

As noted the system 10 is not limited to sensors for photoplethysmographic measurements. Additional sensors can be added, such as temperature sensors, accelerometers, and other physiologic and environmental sensors. These sensors can have their data utilized by the controller 16 to validate the other obtained data of sensors, and vice versa. The additional sensors if any need not operate completely alone. For example a visible light can be an added sensor which is combined with a time elapsed camera on the top of the chamber 12, and together will form a time stamped motion tracking sensor unit.

The system 10 of FIG. 2 further includes two independent fluid injection lines as described above. The system may further include a clip or line holding member (not shown) supported from the rotary bearing 42 to help keep the wires and lines away from the animal to the greatest extent possible. The holding member may be formed of a flexible member.

The advantages of the present system include the application of pulse oximetry to a sealed full body plethysmographic chamber. A further advantage is the application of pulse oximetry from sensor 30 with other physiologic measurements from sensor 50 and adding dual fluid/gas swivel lines 54 to a sealed full body plethysmographic chamber 12. The system passes electrical lines, 12 or more, and two fluid lines into a sealed chamber. The system provides the ability to counter-rotate all lines externally without having to unseal the chamber 12.

The system 10 uses a method of sealing the chamber using a sealed bearing to allow rotation with a pass-thru plug in the inner bearing race to pass two groups of wires (each shown schematically as line 46) and two fluid lines 44. It may be possible to pass additional electrical and/or fluid lines through the bearing 42 as desired, but the system 10 as shown should accommodate a very large number of desired uses. Further with regard to the position of the lines 44 and 46 through the inner plate of the bearing 42, it is expected to have them position at radial spaced locations rather than across a diametrical line as schematically illustrated.

The system 10 may use the wire insulation jacket for wiring groups for each line 46 to help effect a seal through the inner bearing race. The system passes the fluid lines into the chamber whereby they can be easily connected and disconnected on both the inside and the outside of the chamber lid via the connecting nubs. The system 10 may further provides an attaching point protruding from the bearing inner race to support a mechanism for retracting wires up and away from the animal to prevent chewing, such as through rubber bands.

Whereas particular embodiments of the invention have been described above for purposes of illustration, it will be evident to those skilled in the art that numerous variations of the details of the present invention may be made without departing from the spirit and scope of the present invention.

What is claimed is:

1. A full body plethysmographic chamber including a noninvasive photoplethysmographic sensor within the chamber.

2. The full body plethysmographic chamber according to claim 1 further including an electrical line commutator with wires extending from the electrical line commutator to the photoplethysmographic sensor configured for allowing rotation of the wires.

3. The full body plethysmographic chamber according to claim 2 wherein the photoplethysmographic sensor is configured to be mounted on the neck of the animal.

4. The full body plethysmographic chamber according to claim 3 wherein a sealed bearing is positioned between the commutator and the photoplethysmographic sensor, and wherein the wires extending from the commutator to the photoplethysmographic sensor extends through the sealed bearing.

5. The full body plethysmographic chamber according to claim 4 further including at least one fluid/gas line extending to the animal within the chamber.

6. The full body plethysmographic chamber according to claim 5 wherein the fluid/gas line extends through the sealed bearing.

7. The full body plethysmographic chamber according to claim 6 further including a fluid line commutator coupled to the fluid lines.

8. The full body plethysmographic chamber according to claim 7 further including at least one additional physiologic sensor coupled to the animal.

9. The full body plethysmographic chamber according to claim 8 further including wires extending from each additional physiologic sensor to the electrical line commutator.

10. The full body plethysmographic chamber according to claim 9 wherein each physiologic sensor and each fluid line can be selectively coupled and decoupled from the sealed bearing.

11. A method of obtaining physiologic parameters of an animal comprising the steps of:
Placing the animal in a sealed full body plethysmographic chamber;
Obtaining pulmonary parameters of the animal from the body plethysmographic chamber with a first sensor platform; and
Simultaneously obtaining pulse oximetry data from a photoplethysmographic pulse oximetry sensor coupled to the animal.

12. The method according to claim 11 further including an electrical line commutator with wires extending from the electrical line commutator to the photoplethysmographic sensor configured for allowing rotation of the wires.

13. The method according to claim 11 further including mounting the photoplethysmographic sensor on the neck of the animal.

14. The method according to claim 13 wherein a sealed bearing is positioned between the commutator and the photoplethysmographic sensor, and wherein the wires extending from the commutator to the photoplethysmographic sensor extends through the sealed bearing.

15. The method according to claim 11 further including at least one fluid/gas line extending to the animal within the chamber.

16. The method according to claim 15 further including a fluid line commutator coupled to each fluid line.

17. The method according to claim 15 further including the step of simultaneously obtaining further physiologic parameters from at least one additional physiologic sensor coupled directly to the animal within the chamber.

18. A full body plethysmographic chamber including a sealed chamber for receiving an animal; a sensor platform coupled to the chamber for obtaining pulmonary parameters of the animal; at least one additional physiologic sensor coupled directly to the animal within the chamber; and an electrical line commutator with wires extending from the electrical line commutator to each of the physiologic sensors that are coupled directly to the animal with the commutator configured for allowing rotation of the wires.

19. The full body plethysmographic chamber according to claim 18 wherein one physiologic sensors that that is coupled directly to the animal is a photoplethysmographic sensor.

20. The full body plethysmographic chamber according to claim 19 wherein the photoplethysmographic sensor is coupled to the neck of the animal.
